(19) United States
(12) Patent Application Publication
(10) Pub. No.: US 2007/0292383 A1
(43) Pub. Date: Dec. 20, 2007

(54) Use of Surfactant Systems for Reducing Skin-Intrinsic Enzyme Damage

(75) Inventors: Andreas Schepky, Hamburg (DE); Stephan Ruppert, Hamburg (DE); Maren Wilken, Norderstedt (DE); Christian Frese, Hamburg (DE); Ralf Siegner, Pinneberg (DE); Ursula Holtzmann, Hamburg (DE); Martin Kauffeldt, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, HAMBURG (DE)

(21) Appl. No.: 10/579,710

(22) PCT Filed: Oct. 19, 2004
(86) PCT No.: PCT/EP04/52577

§ 371 Date: Mar. 12, 2007

(30) Foreign Application Priority Data
Nov. 19, 2003 (DE) 103 54 115.2

(51) Int. Cl. A61K 31/74

(52) U.S. Cl. 424/78.03

(57) ABSTRACT

A cosmetic and/or dermatological body cleaning preparation with a reduced tendency toward damaging skin-intrinsic enzymes, containing a surfactant system consisting of (1) alkyl ether sulfate, (2) alkyl amidopropyl betain, (3) another surfactant selected from the group of alkyl polyglucosides, ethoxylated triglycerides and the salts of citric acid polyglycol esters, in addition to the use thereof.

The present invention relates to the use of surfactant systems of lauryl ether sulfate, alkylamidopropylbetaine and a further surfactant for reducing skin-intrinsic enzyme damage when cleaning the body or hair.

Definitions

For the purposes of the present specification, skin-intrinsic enzymes are enzymes which are present on the surface of the skin or close to the surface of the skin. Such enzymes can, for example, be hydrolases, such as proteases, esterases, lipases, phosphatases, sulfatases and transglutaminases, but in particular proteases, such as the stratum corneum tryptic enzyme. Tables 1 and 2 show the most important “stratum corneum enzymes” known from the literature.

TABLE 1
Enzymes which degrade desmosomes
and contribute to desquamation

| | Site of | Reaction (barrier | |
|---|---|---|---|
| Enzyme | action | damage) | Literature |
| SCCE | SC (LB) | Cleavage of protein | Lundström, 1991 |
| | | bonds | Suzuki, 1994 |
| | | | Sondell, 1995 |
| | | | Chang-Yi, 1997 |
| Trypsin | SC | Cleavage of protein | Suzuki, 1994 |
| | | bonds | Chang-Yi, 1997 |
| Cathepsins | SG | Filaggrin degradation | Hara, 1993 |
| | | Keratinizing aid | Kawada, 1997 |
| Thiol protease | SC | | Yokozeki, 1987 |

TABLE 2
Enzymes which construct the barrier and
contribute to barrier homeostasis

| | Site of | Reaction (barrier | |
|---|---|---|---|
| Enzyme | action | damage) | Literature |
| Phospholipase A₂ | SG-SC; LB | Release of fatty | Mauro, 1998 |
| | | acids and possibly | Mao-Qiang, |
| | | cholesterol from | 1995 |
| | | cholesterol esters | Elias, 1988 |
| | | | Menon, 1986 |
| Acid lipase | SC, LB | Release of sterols | Menon, 1986 |
| | | | Elias, 1988 |
| Neutral lipase | SC, LB | Sterol - and fatty | Menon, 1986 |
| | | acid - release | |
| | | Regulation of | |
| | | protein kinases | |
| | | (different.) | |
| Sphingomyelinase | SC, LB | Provision of | Menon, 1986 |
| | | ceramides | |
| Ceramidase | SC | None | Jin, 1994 |
| β-Glucocerebrosidase | SC | Conversion of | Holleran, |
| | | glycoceramides | 1992 |
| | | to ceramides | Mauro, 1998 |
| Steroid sulfatase | SC | Release of | Elias, 1988 |
| | | cholesterol from | |
| | | cholesterol sulfate | |
| Sulfatases | SC | Precursor cleavage | Baden, 1980 |

Ammonia lyases play an important role in filaggrin degradation (Kuroda et al., 1979). As do transglutaminases (Polakowska et al., 1991), which are essential for the formation of the “Cornified Envelope”. Phosphatases are the hydrolases with the highest total activity within the stratum corneum.

For the purposes of the present specification, skin-intrinsic enzyme damage means any form of inactivation of these enzymes by denaturation, inhibition or chemical degradation. If enzymes come into contact with surfactants, a denaturation is very often the result. Prottey et al., 1984 quantifies the effect of surfactants on the acidic phosphatase of the stratum corneum (obtained by tape stripping) by measuring the phosphatase activity. In this connection, a reduction in the enzyme activity as a result of denaturation of the enzyme was established. On account of further data, a surfactant sensitivity of most surface-active skin enzymes is to be assumed.

The known products for cleaning the skin comprise, for example, mixtures of lauryl ether sulfate and alkylamidopropylbetaine. The application of such products leads to a partial denaturation of the skin-intrinsic enzymes and thus to skin damage since these enzymes hold an important role physiologically.

The specification WO 2000/11124 discloses washing-active preparations with more than 9% lauryl ether sulfate and N-acylamino acids. When they are applied, they leave behind particularly little surfactant adhering to the surface of the skin and thus counter skin damage as a result of surfactant loading. Surfactant combinations according to the invention are not disclosed.

The specification EP 1210933 discloses skin conditioners which comprise ammonium salts or ions thereof and R1R2R3C—CR4R5-NR6R7, where R1, R2 and R3 are each H, OH, lower alkyl, phosphoryloxy, aryl, R4 and R5 are each H, OH, lower alkyl, phosphoryloxy, aryl, or R4 and R5 together form a carbonyl group;

R6 and R7 are each H, lower alkyl, or else R6 and R2 are alkylene groups which, together with the C atom carrying them, form a five-membered ring. Surfactant combinations according to the invention, on the other hand, are not disclosed.

For the person skilled in the art, it has been found, in an unforeseeable manner, that cosmetic and/or dermatological body cleaning preparation with a reduced tendency toward damaging skin-intrinsic enzymes, comprising a surfactant system of (1) alkyl ether sulfate, (2) alkylamidopropylbetaine, (3) a further surfactant chosen from the group consisting of alkyl polyglucosides, ethoxylated triglycerides and salts of citric acid alkyl polyglycol esters overcome the shortcomings of the prior art. Such preparations or the application thereof brings about reduced skin flakiness, reduced skin roughness, improved skin moisture, improved barrier integrity and function, improved skin integrity and elasticity, improved skin physiology, and improved hair appendage physiology compared with the prior art.

The invention also provides the use of surfactant systems of (1) alkyl ether sulfate, (2) alkylamidopropylbetaine, (3) a further surfactant chosen from the group consisting of alkyl polyglucosides, ethoxylated triglycerides and salts of citric acid alkyl polyglycol ester sulfosuccinates for reducing skin-intrinsic enzyme damage when cleaning the body or hair.

Here, it is preferred if the content of further surfactant (3) is greater than 1% by weight, preferably greater than 1.5% by weight. It is also preferred if the ratio of alkyl ether sulfate to further surfactant is 10:0.5 to 10:5, particularly preferably 10:1 to 10:3 and very particularly preferably 10:1.8 to 10:2.2. As a result, even after frequent showering, the skin-renewing enzymes are protected and thus the natural equilibrium of the skin is supported. It is also preferred if Na cocoyl glutamate is additionally present in the preparations. It is particularly preferred if PEG-7 glyceryl cocoate, disodium PEG-5 lauryl citrate sulfosuccinate or lauryl glucoside is chosen as surfactant (3).

Alkyl polyglucosides are characterized by the structural formula 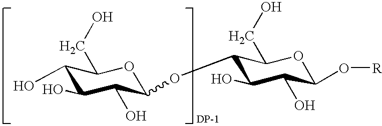
where R is a branched or unbranched alkyl radical having 4 to 24 carbon atoms and where D̅P̅ is an average degree of glucosylation of up to 2.

Particular preference is given to decyl polyglucoside and lauryl polyglucoside, which are sold by Cognis under the trade names Plantacare 2000 or Plantaren 2000 or Plantaren 1200.

Ethoxylated glycerol fatty acid esters (ethoxylated triglycerides) are used in aqueous cleaning formulations for various purposes. Glycerol fatty acid esters (EO 3-12) with low degrees of ethoxylation usually serve as refatting agents for improving the feel on the skin after drying, glycerol fatty acid esters with a degree of ethoxylation of about 30-50 serve as solubility promoters for nonpolar substances such as perfume oils. Glycerol fatty acid esters with high degrees of ethoxylation are used as thickeners. According to the invention, the ethoxylated triglycerides are advantageously chosen from the group of ethoxylated glycerol fatty acid esters, particularly preferably: PEG-10 olive oil glycerides, PEG-11 avocado oil glycerides, PEG-11 cocoa butter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 castor oil, olive oil PEG-7 ester, PEG-6 caprylic acid/capric acid glycerides, PEG-10 olive oil glycerides, PEG-13 sunflower oil glycerides, PEG-7 hydrogenated castor oil, hydrogenated palm kernel oil glyceride PEG-6 ester, PEG-20 corn oil glycerides, PEG-18 glyceryl oleate/cocoate, PEG-40 hydrogenated castor oil, PEG-40 castor oil, PEG-60 hydrogenated castor oil, PEG-60 corn oil glycerides, PEG-54 hydrogenated castor oil, PEG-45 palm kernel oil glycerides, PEG-35 castor oil, PEG-80 glyceryl cocoate, PEG-60 almond oil glycerides, PEG-60 evening primrose glycerides, PEG-200 hydrogenated glyceryl palmate, PEG-90 glyceryl isostearate.

Preferred ethoxylated oils are those with a degree of ethoxylation of 3-15, particularly preferably PEG-7 glyceryl cocoate or PEG-9 cocoglycerides, which are available commercially under the name: Tegosoft GC from Goldschmidt, Cetiol He from Cognis or as Oxypon 401 from Zschimmer & Schwarz. Very particular preference is given to ethoxylated triglycerides with a content of mono- or polyethoxylated glycerol of >20%.

Salts of citric acid alkyl polyglycol ester sulfosuccinates 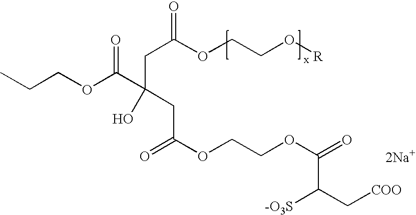
preferably have a degree of ethoxylation x of 3-10, particularly preferably an average degree of ethoxylation of 5. Preferably, R=cocoyl, palmitoyl or lauryl. Particular preference is given to disodium PEG-5 lauryl citrate sulfosuccinate, which is sold by Goldschmidt under the name Rewopol SB CS 50.

The omission of one of the individual constituents adversely affects the unique properties of the overall composition. All of the stated constituents of the preparations according to the invention are therefore expediently required in order to carry out the invention.

Preparations according to the invention can also comprise surfactants. Surfactants are amphiphilic substances which can dissolve organic, nonpolar substances in water. As the result of their specific molecular structure with at least one hydrophilic molecular moiety and one hydrophobic molecular moiety, they provide for a reduction in the surface tension of water, wetting of the skin, easier soil removal and dissolution, easy rinsing and—if desired—for foam regulation.

The hydrophilic moieties of a surfactant molecule are mostly polar functional groups, for example —COO⁻, —OSO₃²⁻, —SO₃⁻, while the hydrophobic moieties are generally nonpolar hydrocarbon radicals. Surfactants are generally classified according to the type and charge of the hydrophilic molecular moiety. Here, four groups can be differentiated:
anionic surfactants,
cationic surfactants,
amphoteric surfactants and
nonionic surfactants.

Anionic surfactants usually have, as functional groups, carboxylate, sulfate or sulfonate groups. In aqueous solution, they form negatively charged organic ions in an acidic or neutral medium. Cationic surfactants are characterized almost exclusively by the presence of a quaternary ammonium group. In aqueous solution, they form positively charged organic ions in an acidic or neutral medium. Amphoteric surfactants contain both anionic and cationic groups and accordingly in aqueous solution exhibit the behavior of anionic or cationic surfactants depending on the pH. In a strongly acidic medium, they have a positive charge, and in an alkaline medium a negative charge. By contrast, in the neutral pH range, they are zwitterionic, as the example below is intended to illustrate:

| | | |
|---|---|---|
| RNH₂⁺CH₂CH₂COOH X⁻ | (at pH = 2) | X⁻ = any anion, e.g. Cl⁻ |
| RNH₂⁺CH₂CH₂COO⁻ | (at pH = 7) | |
| RNHCH₂CH₂COO⁻ | (at pH = 12) | B⁺ = any cation, e.g. Na⁺ |

Typical nonionic surfactants are polyether chains. Nonionic surfactants do not form ions in aqueous medium.

A. Anionic Surfactants

Anionic surfactants to be used advantageously are

Acylamino acids (and salts thereof), such as

1. acyl glutamates, for example sodium acyl glutamate, di-TEA-palmitoyl aspartate and sodium caprylic/capric glutamate,
2. acylpeptides, for example palmitoyl-hydrolyzed milk protein, sodium cocoyl-hydrolyzed soya protein and sodium/potassium cocoyl-hydrolyzed collagen,
3. sarcosinates, for example myristoyl sarcosine, TEA-lauroyl sarcosinate, sodium lauroyl sarcosinate and sodium cocoyl sarcosinate,
4. taurates, for example sodium lauroyl taurate and sodium methyl cocoyl taurate,
5. acyl lactylates, lauroyl lactylate, caproyl lactylate
6. alaninates
   Carboxylic acids and derivatives, such as
1. carboxylic acids, for example lauric acid, aluminum stearate, magnesium alkanolate and zinc undecylenate,
2. ester carboxylic acids, for example calcium stearoyl lactylate, laureth-6 citrate and sodium PEG-4 lauramide carboxylate,
3. ether carboxylic acids, for example sodium laureth-13 carboxylate and sodium PEG-6 cocamide carboxylate,
   Phosphoric esters and salts, such as, for example, DEA-oleth-10 phosphate and dilaureth-4 phosphate,
   Sulfonic acids and salts, such as
1. acyl isethionates, e.g. sodium/ammonium cocoyl isethionate,
2. alkylarylsulfonates,
3. alkylsulfonates, for example sodium cocomonoglyceride sulfate, sodium C₁₂₋₁₄ olefinsulfonate, sodium lauryl sulfoacetate and magnesium PEG-3 cocamide sulfate,
4. sulfosuccinates, for example dioctyl sodium sulfosuccinate, disodium laureth sulfosuccinate, disodium lauryl sulfosuccinate, disodium undecylenamido-MEA sulfosuccinate and PEG-5 lauryl citrate sulfosuccinate.
   And
   Sulfuric esters, such as
1. alkyl ether sulfate, for example sodium, ammonium, magnesium, MIPA, TIPA laureth sulfate, sodium myreth sulfate and sodium C₁₂₋₁₃-pareth sulfate,
2. alkyl sulfates, for example sodium, ammonium and TEA lauryl sulfate.
   B. Cationic Surfactants

Cationic surfactants to be used advantageously are
1. alkylamines,
2. alkylimidazoles,
3. ethoxylated amines and
4. quaternary surfactants,
5. ester quats.

Quaternary surfactants contain at least one N atom which is covalently bonded to 4 alkyl and/or aryl groups. Irrespective of the pH, this leads to a positive charge. Advantageous quaternary surfactants are alkylbetaine, alkylamidopropylbetaine and alkylamidopropylhydroxysulfaine. For the purposes of the present invention, cationic surfactants can also preferably be chosen from the group of quaternary ammonium compounds, in particular benzyltrialkylammonium chlorides or bromides, such as, for example, benzyldimethylstearylammonium chloride, also alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl- or cetylpyrimidinium chloride, imidazoline derivatives and compounds with cationic character, such as amine oxides, for example alkyldimethylamine oxides or alkylaminoethyldimethylamine oxides. In particular, cetyltrimethylammonium salts are to be used advantageously.

C. Amphoteric Surfactants

Amphoteric surfactants to be used advantageously are
1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,
2. N-alkylamino acids, for example aminopropylalkylglutamide, alkylaminopropionic acid, sodium alkylimidodipropionate and lauroamphocarboxyglycinate.
   D. Nonionic Surfactants

Nonionic surfactants to be used advantageously are
1. alcohols,
2. alkanolamides, such as cocamides MEA/DEA/MIPA,
3. amine oxides, such as cocoamidopropylamine oxide,
4. esters which are formed by esterification of carboxylic acids with ethylene oxide, glycerol, sorbitan or other alcohols,
5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside,
6. sucrose esters, sucrose ethers
7. polyglycerol esters, diglycerol esters, monoglycerol esters
8. methyl glucose esters, esters of hydroxyl acids

Also advantageous is the use of a combination of anionic and/or amphoteric surfactants with one or more nonionic surfactants.

Cleaning preparations according to the invention are advantageously in the form of gels and comprise one or more gel formers and/or hydrocolloids.

“Hydrocolloid” is the technical abbreviation for the per se more correct name “hydrophilic colloid”. Hydrocolloids are macromolecules which have a largely linear configuration and have intermolecular forces of interaction which permit secondary and primary valency bonds between the individual molecules and thus the formation of a reticular structure. They are sometimes water-soluble natural or synthetic polymers which form gels or viscous solutions in aqueous systems. They increase the viscosity of water by either binding water molecules (hydration), or by absorbing and encapsulating the water into their interwoven macromolecules, at the same time as restricting the mobility of the water. Such water-soluble polymers represent a large group of chemically very different natural and synthetic polymers whose common feature is their solubility in water or aqueous media. A prerequisite for this is that these polymers have a number of hydrophilic groups sufficient for solubility in water and are not too greatly crosslinked. The hydrophilic groups may be nonionic, anionic or cationic in nature, for example as follows:

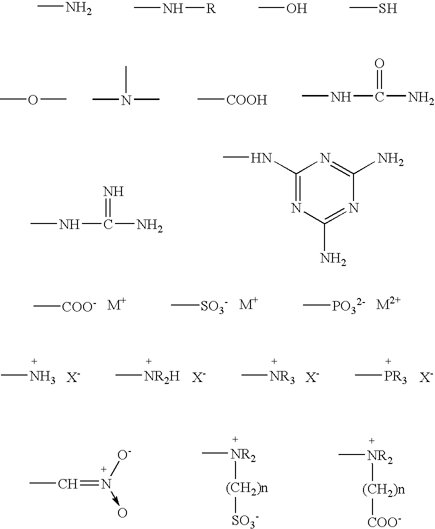

The group of cosmetically and dermatologically relevant hydrocolloids can be divided as follows into:

organic, natural compounds, such as, for example, agar agar, carrageen, tragacanth, gum arabic, alginates, pectins, polyoses, guar flour, carob bean flour, starch, dextrins, gelatin, casein,

organic, modified natural substances, such as, for example, carboxymethylcellulose and other cellulose ethers, hydroxyethylcellulose and hydroxypropylcellulose and the like,

organic, completely synthetic compounds, such as, for example, polyacrylic and polymethacrylic compounds, vinyl polymers, polycarboxylic acids, polyethers, polyimines, polyamides,

inorganic compounds, such as, for example, polysilicic acids, clay minerals, such as montmorillonite, zeolites, silicas.

Hydrocolloids preferred according to the invention are, for example, methylcelluloses, which is the term used to refer to the methyl ethers of cellulose. They are characterized by the following structural formula 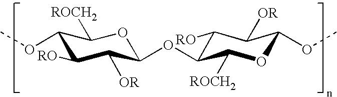
in which R may be a hydrogen or a methyl group.

Of particular advantage for the purposes of the present invention are the cellulose mixed ethers, which are generally likewise referred to as methylcelluloses and which, besides a dominant content of methyl groups, additionally contain 2-hydroxyethyl, 2-hydroxypropyl or 2-hydroxybutyl groups. Particular preference is given to (hydroxypropyl)methylcelluloses, for example those available under the trade name Methocel E4M from Dow Chemical Comp.

Also advantageous according to the invention is sodium carboxymethylcellulose, the sodium salt of the glycolic acid ether of cellulose for which R in structural formula I may be a hydrogen and/or CH₂—COONa. Particular preference is given to the sodium carboxymethylcellulose available under the trade name Natrosol Plus 330 CS from Aqualon, which is also referred to as cellulose gum.

Also preferred for the purposes of the present invention is xanthan (CAS No. 11138-66-2), also called xanthan gum, which is an anionic heteropolysaccharide which is generally formed by fermentation from corn sugar and is isolated as the potassium salt. It is produced by Xanthomonas campestris and a few other species under aerobic conditions with a molecular weight of 2×106 to 24×106. Xanthan is formed from a chain with β-1,4-bonded glucose (cellulose) with side chains. The structure of the subgroups consists of glucose, mannose, glucuronic acid, acetate and pyruvate. Xanthan is the name for the first microbial anionic heteropolysaccharide. It is produced by Xanthomonas campestris and a few other species under aerobic conditions with a molecular weight of 2-15 106. Xanthan is formed from a chain with β-1,4-bonded glucose (cellulose) with side chains. The structure of the subgroups consists of glucose, mannose, glucuronic acid, acetate and pyruvate. The number of pyruvate units determines the viscosity of the xanthan. Xanthan is produced in two-day batch cultures with a yield of 70-90%, based on carbohydrate used. Yields of 25-30 g/l are achieved here. After killing the culture, work-up takes place by precipitation with e.g. 2-propanol. Xanthan is then dried and ground.

An advantageous gel former for the purposes of the present invention is also carrageen, a gel-forming extract with a similar structure to that of agar from north Atlantic red algae which belong to the florideae (Chondrus crispus and Gigartina stellata).

The name carrageen is often used for the dried algae product and carrageenan for the extract from this. The carrageen precipitated from the hot-water extract of the algae is a colorless to sand-colored powder with a molecular weight range of 100 000-800 000 and a sulfate content of about 25%. Carrageen, which is very readily soluble in warm water, upon cooling forms a thixotropic gel, even if the water content is 95-98%. The strength of the gel is brought about by the double helix structure of the carrageen. In the case of carrageenan, a distinction is made between three main constituents: the gel-forming κ-fraction consists of D-galactose-4-sulfate and 3,6-anhydro-α-D-galactose, which are alternately glycosidically bonded in the 1,3 and 1,4 position (in contrast, agar comprises 3,6-anhydro-α-L-galactose). The nongelling λ-fraction is composed of 1,3-glycosidically bonded D-galactose-2-sulfate and 1,4-bonded D-galactose-2,6-disulfate radicals and is readily soluble in cold water. The 1-carrageenan formed from D-galactose-4-sulfate in 1,3 bonding and 3,6-anhydro-α-D-galactose-2-sulfate in 1,4 bonding is both water-soluble and also gel-forming. Other carrageen types are likewise referred to with Greek letters: α, β, γ, μ, ν, ξ, π, ω, χ. The type of cations present (K+, NH₄+, Na+, Mg2+, Ca2+) also influences the solubility of the carrageens.

Polyacrylates are gelling agents likewise to be used advantageously for the purposes of the present invention. Polyacrylates advantageous according to the invention are acrylate-alkyl acrylate copolymers, in particular those chosen from the group of so-called carbomers or carbopols (Carbopol® is actually a registered trademark of NOVEON Inc.). In particular, the acrylate-alkyl acrylate copolymers advantageous according to the invention are characterized by the following structure: 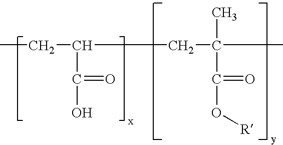
where R′ is a long-chain alkyl radical and x and y represent numbers which symbolize the respective stoichiometric proportion of the particular comonomers.

According to the invention, particular preference is given to acrylate copolymers and/or acrylate-alkyl acrylate copolymers which are available under the trade names Carbopol® 1382, Carbopol® 981 and Carbopol® 5984, Aqua SF-1 from NOVEON Inc. and as Aculyn® 33 from International Specialty Products Corp.

Also advantageous are copolymers of C10-30-alkyl acrylates and one or more monomers of acrylic acid, of methacrylic acid or esters thereof, which are crosslinked with an allyl ether of sucrose or an allyl ether of pentaerythritol.

Compounds which bear the INCI name “Acrylates/C 10-30 Alkyl Acrylate Crosspolymer” are advantageous. Particularly advantageous are those available under the trade names Pemulen TR1 and Pemulen TR2 from NOVEON Inc.

Also advantageous are compounds which the INCI name “acrylates/C12-24 pareth-25 acrylate copolymer” (available under the trade names Synthalen® W2000 from 3V Inc.), which the INCI name “acrylates/steareth-20 methacrylate copolymer” (available under the trade names Aculyn® 22 from International Specialty Products Corp.), which the INCI name “acrylates/steareth-20 itaconate copolymer” (available under the trade names Structure 2001® from National Starch), which the INCI name “acrylates/aminoacrylates/C10-30 alkyl PEG-20 itaconate copolymer” (available under the trade names Structure Plus® from National Starch) and similar polymers.

The total amount of one or more hydrocolloids in the finished cosmetic or dermatological preparations is advantageously chosen to be less than 1.5% by weight, preferably between 0.1 and 1.0% by weight, based on the total weight of the preparations.

For the purposes of the present invention, it is advantageous if the content of one or more polyacrylates in the cosmetic or dermatological cleaning emulsion is chosen from the range from 0.5 to 4% by weight, very particularly advantageously from 0.7 to 2% by weight, in each case based on the total weight of the preparations.

It is also advantageous to add complexing agents to the preparations according to the invention. The complexing agents are advantageously chosen from the group consisting of ethylenediaminetetraacetic acid (EDTA) and anions thereof, nitrilotriacetic acid (NTA) and anions thereof, hydroxyethylenediaminotriacetic acid (HOEDTA) and anions thereof, diethyleneaminopentaacetic acid (DPTA) and anions thereof, trans-1,2-diaminocyclohexanetetraacetic acid (CDTA) and anions thereof, tetrasodium iminodisuccinate, trisodium ethylenediamine disuccinate.

In addition, conditioning auxiliaries may be present in the cosmetic cleaning compositions, e.g. in amounts of from 0.001 to 10% by weight, based on the total weight of the preparations.

Preferred conditioning auxiliaries include cationic polymers, which provide for an improvement in the care properties on the hair.

These include cationic cellulose derivatives synthesized on the basis of hydroxycellulose with a trimethylammonium-substituted epoxide. These substances are known under the name Polyquaternium-10 and are commercially available, for example, as Polymer JR 400 from Union Carbide Cooperation.

Further substances e.g.: cationic polysaccharides particularly modified guar derivatives, known under the name JAGUAR C13S and sold by Meyhall; homopolymers and copolymers based on (meth)acryloyloxyethyltrimethylammonium salt with the trade name Salcare SC92 or Salcare SC95 obtainable from Allied Colloids; polymers based on the monomer diallyldimethylammonium chloride, such as polyquaternium-6 as homopolymer with the trade name Salcare SC30 and polyquaternium-7 as copolymer with acrylamide under the trade name Salcare SC10; polyquaternium-47 as copolymer of acrylic acid, methacrylate and methacrylamidopropyltrimonium chloride with the trade name Merquat 2001N from Calgon; copolymers of vinylpyrrolidone and vinylmethylimidazolium salt, such as polyquaternium-44 obtainable as Luviquat Care from BASF; terpolymers of vinylpyrrolidone, dimethylaminopropylmethacrylamide and alkyldimethylaminopropylmethacrylamidoammonium salts under the trade name Styleze W-20 from ISP.

Preservatives permitted in food technology, which are listed below with their E number, are to be used advantageously according to the invention.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | p-Hydroxybenzoic ethyl ester Na salt |
| E 216 | n-Propyl p-hydroxybenzoate |
| E 217 | p-Hydroxybenzoic n-propyl ester Na salt |
| E 218 | Methyl p-hydroxybenzoate |
| E 219 | p-hydroxybenzoic methyl ester Na salt |
| E 220 | Sulfur dioxide |
| E 221 | Sodium sulfite |
| E 222 | Sodium hydrogen sulfite |
| E 223 | Sodium disulfite |
| E 224 | Potassium disulfite |
| E 226 | Calcium sulfite |
| E 227 | Calcium hydrogen sulfite |
| E 228 | Potassium hydrogen sulfite |
| E 230 | Biphenyl (diphenyl) |
| E 231 | Orthophenylphenol |
| E 232 | Sodium orthophenylphenoxide |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also suitable according to the invention are preservatives or preservative auxiliaries customary in cosmetics dibromodicyanobutane (2-bromo-2-bromomethylglutarodinitrile), 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol, DMDM hydantoin, IPBC (formaldehyde donor).

Also suitable as preservatives are phenyl hydroxyalkyl ethers, in particular the compound known under the name phenoxyethanol, on account of their bactericidal and fungicidal effects on a number of microorganisms.

Other antimicrobial agents are likewise suitable to be incorporated into the preparations according to the invention. Advantageous substances are, for example, 2,4,4′-trichloro-2′-hydroxydiphenyl ether (Irgasan), 1,6-di(4-chlorophenylbiguanido)hexane (chlorhexidine), 3,4,4′-trichlorocarbanilide, quaternary ammonium compounds, oil of cloves, mint oil, thyme oil, triethyl citrate, farnesol (3,7,11-trimethyl-2,6,10-dodecatrien-1-ol), and the active ingredients or active ingredient combinations described in the patent laid-open specifications DE-37 40 186, DE-39 38 140, DE-42 04 321, DE-42 29 707, DE-43 09 372, DE-44 11 664, DE-195 41 967, DE-195 43 695, DE-195 43 696, DE-195 47 160, DE-196 02 108, DE-196 02 110, DE-196 02 111, DE-196 31 003, DE-196 31 004 and DE-196 34 019 and the patent specifications DE-42 29 737, DE-42 37 081, DE-43 24 219, DE-44 29 467, DE-44 23 410 and DE-195 16 705. Sodium hydrogencarbonate is also to be used advantageously.

For all this, it is possible in specific cases for the abovementioned concentration data to be slightly exceeded or fallen below and preparations according to the invention nevertheless to be obtained. In view of the greatly scattered diversity of suitable components of such preparations, this is not unexpected for the person skilled in the art, so that he knows that such excesses or deficits do not depart from the essence of the present invention.

The examples below are intended to illustrate the present invention without limiting it. The numerical values in the examples are percentages by weight, based on the total weight of the particular preparations.

### EXAMPLES

| | 1 | 2 | 3 | 4 | 5 |
|---|---|---|---|---|---|
| Sodium laureth sulfate | 13% | 11% | 9.75% | 5.5% | 10% |
| Cocoamidopropylbetaine | 1.9% | 3.3% | 3.8% | 4% | 1% |
| PEG-7 glyceryl cocoate | 1% | 1.5% | 2% | 0.8% | — |
| Sodium cocoyl glutamate | 1.25% | 0.75% | 2.0% | — | 2.8% |
| PEG-40 hydrogenated castor oil | 0.50% | 0.50% | 0.5% | 0.4% | — |
| PEG-90 glyceryl isostearate | | | | | |
| PEG-100 hydrogenated glyceryl palmitate | 0.50% | 0.50% | 0.5% | 0.9% | 0.4% |
| Almond oil | — | 0.2% | 0.2% | — | — |
| Polyquaternium-10 | 0.2% | — | 0.2% | 0.1% | 0.2% |
| Sodium benzoate | 0.45% | 0.45% | 0.45% | 0.4% | 0.4% |
| Sodium salicylate | 0.20% | 0.20% | 0.2% | 0.2% | 0.2% |
| Citric acid | 0.50% | 0.50% | 0.5% | 0.5% | 0.4% |
| Perfume | q.s. | q.s. | q.s. | q.s. | — |
| Water | ad 100 | ad 100 | ad 100 | ad 100 | ad 100 |
| | 6 | 7 | 8 | 9 | 10 |
| Sodium laureth sulfate | 13% | 11% | 9.75% | 5.5% | 10% |
| Cocoamidopropylbetaine | 1.9% | 3.3% | 3.8% | 4% | 1% |
| Decyl polyglucoside | 1.5% | 3% | 2% | 0.3% | 3% |
| Sodium cocoyl glutamate | 1.25% | 0.75% | 2.0% | — | 2.8% |
| PEG-40 hydrogenated castor oil | 0.50% | 0.50% | 0.5% | 0.4% | — |
| PEG-90 glyceryl isostearate | | | | | |
| PEG-100 hydrogenated glyceryl palmitate | 0.50% | 0.50% | 0.5% | 0.9% | 0.4% |
| Almond oil | — | 0.2% | 0.2% | — | — |
| Polyquaternium-10 | 0.2% | — | 0.2% | 0.1% | 0.2% |
| Sodium benzoate | 0.45% | 0.45% | 0.45% | 0.4% | 0.4% |
| Sodium salicylate | 0.20% | 0.20% | 0.2% | 0.2% | 0.2% |
| Citric acid | 0.50% | 0.50% | 0.5% | 0.5% | 0.4% |
| Perfume | q.s. | q.s. | q.s. | q.s. | — |
| Water | ad 100 | ad 100 | ad 100 | ad 100 | ad 100 |
| | 11 | 12 | 13 | 14 | 15 |
| Sodium laureth sulfate | 13% | 11% | 9.75% | 5.5% | 10% |
| Cocoamidopropylbetaine | 1.9% | 3.3% | 3.8% | 4% | 1% |
| PEG-5 laurylcitrate sulfosuccinate | 2% | 1% | 1.2% | 1% | 1% |
| Sodium cocoyl glutamate | 1.25% | 0.75% | 2.0% | — | 2.8% |
| PEG-40 hydrogenated castor oil | 0.50% | 0.50% | 0.5% | 0.4% | — |
| PEG-90 glyceryl isostearate | | | | | |
| PEG-100 hydrogenated glyceryl palmitate | 0.50% | 0.50% | 0.5% | 0.9% | 0.4% |
| Almond oil | — | 0.2% | 0.2% | — | — |
| Polyquaternium-10 | 0.2% | — | 0.2% | 0.1% | 0.2% |
| Sodium benzoate | 0.45% | 0.45% | 0.45% | 0.4% | 0.4% |
| Sodium salicylate | 0.20% | 0.20% | 0.2% | 0.2% | 0.2% |
| Citric acid | 0.50% | 0.50% | 0.5% | 0.5% | 0.4% |
| Perfume | q.s. | q.s. | q.s. | q.s. | — |
| Water | ad 100 | ad 100 | ad 100 | ad 100 | ad 100 |

| | 12 | 13 | 14 | 15 | 16 | 17 |
|---|---|---|---|---|---|---|
| Sodium laureth sulfate | 10.0% | 8.0% | 10% | | 5% | 10.0% |
| Sodium pareth sulfate | | | | 9% | | |
| Sodium myreth sulfate | 1% | | 3.0% | | | 3.0% |
| Cocoamidopropylbetaine | 5% | 5% | 3.0% | 5% | 4.0% | 3.0% |
| Sodium cocoyl glutamate | 2% | 2.0% | 1.5% | 1% | 2.5% | 1.5% |
| Decyl glucoside | | | | | 1.5% | |
| PEG-40 hydrogenated castor oil | 0.5% | 0.5% | 0.5% | 1% | 1% | 0.5% |
| PEG-7 glyceryl cocoate | 2.0% | 2.3% | 2.0% | 1.5% | 1% | 2.0% |
| Glycerol | | 0.3% | 0.5% | | 5.0% | |
| PEG-200 hydrogenated glyceryl | 0.5% | 0.3% | 0.5% | 0.1% | 0.3% | 0.5% |
| palmitate | | | | | | |
| PEG-90 glyceryl isostearate | | 0.3% | | 0.5% | | |
| Laureth-2 | | 0.1% | | 0.1% | | |
| Sodium chloride | 1.0% | 1.0% | 2.0% | 1.0% | 1.0% | 2.0% |
| Trisodium EDTA | 0.2% | | 0.2% | | 0.2% | 0.2% |
| Tetrasodium iminodisuccinate | | 0.8% | | | | |
| Polyquaternium-10 | 0.2% | | | 0.2% | | 0.1% |
| Guar hydroxypropyltrimonium chloride | | | | 0.3% | | 0.2% |
| Benzophenone-4 | | | 0.1% | | 0.1% | 0.1% |
| Glycol distearate | | 0.6% | | | | |
| Laureth-4 | | 0.3% | | | | |
| Styrene/acrylate copolymer | 1.0% | | | 1.0% | | |
| Alcohol denat. | | 1.0% | | | | |
| Plant extracts | | | | 0.2% | | |
| Natural oils | 0.2% | | | | | |
| Preservatives | q.s. | q.s. | q.s. | q.s. | q.s. | q.s. |
| Dyes | q.s. | q.s. | q.s. | q.s. | q.s. | q.s. |
| Citric acid | q.s. | q.s. | q.s. | q.s. | q.s. | q.s. |
| Perfume | q.s. | q.s. | q.s. | q.s. | q.s. | q.s. |
| Water | ad 100 | ad 100 | ad 100 | ad 100 | ad 100 | ad 100 |
| | 18 | 19 | 20 | 21 | 22 | 23 |
| Sodium laureth sulfate | 9.0% | 10.0% | 7.5% | 8.0% | 6% | |
| Sodium pareth sulfate | | | 2% | | | 9.5% |
| Cocoamidopropylbetaine | 1.0% | 7.0% | 3.25% | 3.5% | 3.5% | 3.0% |
| Sodium cocoyl glutamate | 2.0% | 0.5% | 0.2% | 0.5% | 0.5% | 2.0% |
| Lauryl glucoside | | 0.5% | 2.0% | 4.0% | 3% | |
| Disodium PEG-5 laurylcitrate sulfo- | 2.5% | | | | | 2.5% |
| succinate | | | | | | |
| PEG-40 hydrogenated castor oil | 0.5% | 0.5% | 0.5% | 0.5% | 0.5% | 0.5% |
| PEG-7 glyceryl cocoate | | 0.3% | 0.2% | 0.2% | 0.2% | |
| Glyceryl laurate | | 0.2% | | | | |
| PEG-200 hydrogenated glyceryl | 0.2% | 0.3% | 0.5% | | 2.5% | 0.2% |
| palmitate | | | | | | |
| PEG-90 glyceryl isostearate | 0.2% | | | 0.3% | 0.2% | 0.2% |
| Laureth-2 | 0.1% | | | 0.1% | 0.1% | 0.1% |
| PEG-120 methyl glucose dioleate | | | 0.5% | | | |
| Sodium chloride | 1.0% | 1.1% | 1.0% | | 0.5% | 1.0% |
| Trisodium EDTA | | | | | 1.0% | |
| Tetrasodium iminodisuccinate | 1.0% | | | | | 1.0% |
| Polyquaternium-10 | | | | 0.1% | | |
| Guar hydroxypropyltrimonium chloride | | | | 0.2% | | |
| Benzophenone-4 | 0.1% | | | | | 0.1% |
| Glycol distearate | | 0.8% | 0.6% | | 0.6% | |
| Glycerol | | 0.4% | 0.3% | | 0.3% | |
| Laureth-4 | | 0.4% | 0.3% | | 0.3% | |
| Styrene/acrylate copolymer | | | | 1.0% | | |
| Plant extracts | | | 0.05% | | | |
| Natural oils | | | | | 0.2% | |
| Preservatives | q.s. | q.s. | q.s. | q.s. | q.s. | q.s. |
| Dyes | q.s. | q.s. | q.s. | q.s. | q.s. | q.s. |
| Citric acid | q.s. | q.s. | q.s. | q.s. | q.s. | q.s. |
| Perfume | q.s. | q.s. | q.s. | q.s. | q.s. | q.s. |
| Water | ad 100 | ad 100 | ad 100 | ad 100 | ad 100 | ad 100 |

1-6. (canceled)

7. A cosmetic or dermatological body cleaning preparation with a reduced tendency toward damaging skin-intrinsic enzymes, comprising a surfactant system of:
(1) alkyl ether sulfate,
(2) alkylamidopropylbetaine, and
(3) at least one additional surfactant selected from the group consisting of alkyl polyglucosides, ethoxylated triglycerides and salts of citric acid alkyl polyglycol esters.

8. The preparation as claimed in claim 7, wherein the content of surfactant (3) is greater than 1% by weight.

9. The preparation as claimed in claim 7, wherein the content of surfactant (3) is greater than 1.5% by weight.

10. The preparation as claimed in claim 7, wherein the weight ratio of alkyl ether sulfate to surfactant (3) is 10:0.5 to 10:5.

11. The preparation as claimed in claim 7, wherein the weight ratio of alkyl ether sulfate to surfactant (3) is 10:1 to 10:3.

12. The preparation as claimed in claim 7, wherein the weight ratio of alkyl ether sulfate to surfactant (3) is 10:1.8 to 10:2.2.

13. The preparation as claimed in claim 7, further comprising Na cocoyl glutamate.

14. The preparation as claimed in claim 7, wherein the surfactant (3) includes at least one salt of a citric acid alkyl polyglycol ester sulfosuccinate.

15. The preparation as claimed in claim 7, wherein the surfactant (3) is at least one surfactant selected from the group consisting of PEG-7 glyceryl cocoate, disodium PEG-5 lauryl citrate sulfosuccinate and lauryl glucoside.

16. A method of cleaning the body or hair, comprising applying to the body or hair a cosmetic or dermatological body cleaning preparation comprising a surfactant system of:
(1) alkyl ether sulfate,
(2) alkylamidopropylbetaine, and
(3) at least one additional surfactant selected from the group consisting of alkyl polyglucosides, ethoxylated triglycerides and salts of citric acid alkyl polyglycol esters.

17. The method as claimed in claim 16, wherein the content of surfactant (3) is greater than 1% by weight.

18. The method as claimed in claim 16, wherein the content of surfactant (3) is greater than 1.5% by weight.

19. The method as claimed in claim 16, wherein the weight ratio of alkyl ether sulfate to surfactant (3) is 10:0.5 to 10:5.

20. The method as claimed in claim 16, wherein the weight ratio of alkyl ether sulfate to surfactant (3) is 10:1 to 10:3.

21. The method as claimed in claim 16, wherein the weight ratio of alkyl ether sulfate to surfactant (3) is 10:1.8 to 10:2.2.

22. The method as claimed in claim 16, further comprising Na cocoyl glutamate.

23. The method as claimed in claim 16, wherein the surfactant (3) includes at least one salt of a citric acid alkyl polyglycol ester sulfosuccinate.

24. The method as claimed in claim 16, wherein the surfactant (3) is at least one surfactant selected from the group consisting of PEG-7 glyceryl cocoate, disodium PEG-5 lauryl citrate sulfosuccinate and lauryl glucoside.
